(19) United States
(12) Patent Application Publication
(10) Pub. No.: US 2020/0359892 A1
(43) Pub. Date: Nov. 19, 2020

(54) VIRTUAL CONSULTATION SYSTEMS

(71) Applicant: ROLLINS ENTERPRISES, LLC., Woodland Hills, CA (US)

(72) Inventors: Aaron ROLLINS, Los Angeles, CA (US); Ronald Paul ZELHOF, Palmetto Bay, FL (US)

(21) Appl. No.: 16/503,123

(22) Filed: Jul. 3, 2019

Related U.S. Application Data
(60) Provisional application No. 62/848,448, filed on May 15, 2019.

(51) Int. Cl. A61B 5/00; G06Q 30/02; G06F 3/0488; G06T 19/00

(52) U.S. Cl. CPC A61B 5/0002; G06T 19/006; G06F 3/04886; G06Q 30/0281

(57) ABSTRACT

A virtual consultation panel is disclosed. The virtual consultation panel may include processing circuitry, a camera, communications circuitry, and a display panel large enough to display a life-sized or nearly life-sized view of a remote patient, in a live video feed captured by the patient's own device. The patient's device can be a smart phone, a tablet, a desktop computer, or any other device with a camera and communications circuitry for providing images from the camera to the virtual consultation panel. The virtual consultation panel allows a practitioner such as a surgeon to accurately examine the physical features of the patient using the live video feed. In some circumstances, the patient's device can provide absolute scale information to be used in generating the life-sized or nearly life-sized view of the remote patient.

### CROSS REFERENCE TO RELATED APPLICATIONS

This application claims the benefit of, and priority to, U.S. Provisional Patent Application No. 62/848,448, entitled “VIRTUAL CONSULTATION SYSTEMS AND METHODS” filed on May 15, 2019, which is hereby incorporated by reference in its entirety for all purposes.

### TECHNICAL FIELD

The present disclosure generally relates to remote communications methods and, more particularly, to virtual consultation systems.

### BACKGROUND

The process of physically going to a doctor's office, particularly for a plastic surgery consultation, can be a barrier to services for many. For example, the patient can feel vulnerable in the unfamiliar setting of the doctor's office, or the time and distance of travel to and/or from the doctor's office can be prohibitive. This can be particularly true for patients located remotely (i.e., in a different city or town) from the desired doctor's office.

### SUMMARY

The present disclosure provides virtual consultation panels. In some examples, the virtual consultation panel is provided in a virtual consultation system having one or more virtual consultation panels and a practitioner server. The virtual consultation panels allow surgeons, such as plastic surgeons, to view life-size or nearly life-size video feeds of a patient in a location of the patient's own choosing, such as the patient's own home. The video feeds are captured by the patient's own device, such as a smart phone, a tablet, or the like.

As described in further detail hereinafter, the virtual consultation panels described herein are configured to cooperate with patient devices in a way that allows the consulting surgeon to obtain physical information about the patient, without the need for the patient to be present with the surgeon. In this way, one or more barriers to care are lowered or eliminated using the technological solution of the virtual consultation panel.

According to some aspects of the present disclosure, a virtual consultation panel is provided that includes a substrate having a first side and an opposing second side. The virtual consultation panel also includes a display panel coupled to the first side of the substrate and including an array of display pixels configured to project display light through the substrate in an active mode for the display panel. The virtual consultation panel also includes a structural support member configured to support the substrate and the display panel for viewing of the display light. The virtual consultation panel also includes communications circuitry configured to receive a live video stream from a remote user device of a patient. The virtual consultation panel also includes processing circuitry configured to scale the live video stream to generate a virtual consultation view of the live video stream, for display by the display panel in the active mode. The opposing second side of the substrate comprises a mirrored outer surface that obscures viewing of the display panel, in an inactive mode for the display panel.

According to other aspects of the present disclosure, a virtual consultation panel is provided that includes communications circuitry configured to receive a live video stream from a remote user device of a user, the live video stream including images of at least a portion of the user's body. The virtual consultation panel also includes a display panel configured to display the live video stream, the display having a size that is sufficiently large to display a life-size representation of at least a part of at least the portion of the user's body. The virtual consultation panel also includes processing circuitry configured to generate the life-size representation of at least the part of at least the portion of the body based on information received from the remote user device.

According to other aspects of the present disclosure, a virtual consultation panel is provided that includes a display panel and processing circuitry configured to: receive, from a remote user device, a live video stream including images of a patient; receive, from the remote user device, three-dimensional information associated with the images of the patient; and generate, for display by the display panel, a three-dimensional representation of at least a portion of the patient based on the images and the three-dimensional information. The virtual consultation panel also includes an input component configured to receive an input that simulates a three-dimensional manipulation of the three-dimensional representation. The processing circuitry is further configured to modify the three-dimensional representation of at least a portion of the patient based on the images, the three-dimensional information, and the input.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an example system for virtual consultations, according to aspects of the present disclosure.

FIG. 2 is a schematic illustration of a virtual consultation panel with an inactive display panel, according to aspects of the present disclosure.

FIG. 3 is a schematic illustration of a virtual consultation panel with an active display panel, according to aspects of the present disclosure.

FIG. 4 is a block diagram illustrating an example user device, virtual consultation panel, and practitioner server from the architecture of FIG. 1, according to certain aspects of the disclosure.

FIG. 5 is a schematic illustration of a rear side of a virtual consultation panel, according to aspects of the present disclosure.

FIG. 6 is a schematic illustration of the virtual consultation panel of FIG. 5 with an inactive display panel, according to aspects of the present disclosure.

FIG. 7 is a schematic illustration of the virtual consultation panel of FIG. 5 with an active display panel, according to aspects of the present disclosure.

FIG. 8 is a schematic illustration of a cross-sectional side view of a virtual consultation panel, according to aspects of the present disclosure.

FIG. 9 is a schematic illustration of the cross-sectional side view of the virtual consultation panel of FIG. 8 during a mounting operation, according to aspects of the present disclosure.

FIG. 10 is a schematic illustration of the cross-sectional side view of the virtual consultation panel of FIG. 8 mounted to a wall, according to aspects of the present disclosure.

FIG. 11 is a schematic illustration of a cross-sectional side view of another virtual consultation panel, according to aspects of the present disclosure.

FIG. 12 is a schematic illustration of a cross-sectional side view of another virtual consultation panel, according to aspects of the present disclosure.

FIG. 13 is a schematic illustration of a cross-sectional side view of another virtual consultation panel, according to aspects of the present disclosure.

FIG. 14 is a schematic illustration of a virtual consultation panel displaying a selectable connection option, according to aspects of the present disclosure.

FIG. 15 is a schematic illustration of a virtual consultation panel displaying a live video feed of a patient, according to aspects of the present disclosure.

FIG. 16 is a schematic illustration of a virtual consultation panel displaying menu options for control of the virtual consultation panel, according to aspects of the present disclosure.

FIG. 17 is a schematic illustration of a virtual consultation panel displaying menu options for 3D tools of the virtual consultation panel, according to aspects of the present disclosure.

FIG. 18 is a schematic illustration of a virtual consultation panel displaying a live video feed of a patient and scale information associated with the live video feed, according to aspects of the present disclosure.

FIG. 19 is a flow chart of illustrative operations that may be performed for a virtual consultation using a virtual consultation panel, according to aspects of the present disclosure.

FIG. 20 is a flow chart of illustrative operations that may be performed for 3D display operations during a virtual consultation using a virtual consultation panel, according to aspects of the present disclosure.

FIG. 21 illustrates a cross-sectional side view of a freestanding virtual consultation panel having a support structure, according to aspects of the present disclosure.

FIG. 22 illustrates an electronic system with which one or more implementations of the subject technology may be implemented.

### DETAILED DESCRIPTION

In the following detailed description, numerous specific details are set forth to provide a full understanding of the present disclosure. It will be apparent, however, to one ordinarily skilled in the art, that embodiments of the present disclosure may be practiced without some of the specific details. In other instances, well-known structures and techniques have not been shown in detail so as not to obscure the disclosure.

### General Overview

The present disclosure relates to virtual consultation panels. A virtual consultation panel may include a display panel for displaying a live video feed including images of a remote patient, a camera from capturing a live video feed of a consulting surgeon, and communications and processing circuitry for establishing a two-way video connection between a user device of the remote patient and the virtual consultation panel. During a virtual consultation, various instructions are provided to the patient, via the virtual consultation panel and the user device, for performance of actions for the virtual consultation. The virtual consultation panel may include a scale processing engine to process scale information from the user device, and to scale displayed images and/or provide scale indicators for display. In this way, the virtual consultation panel provides tools that allow the consulting surgeon to view and/or determine the actual size of the patient and portions of the patient under consideration for surgery.

### Example System Architecture

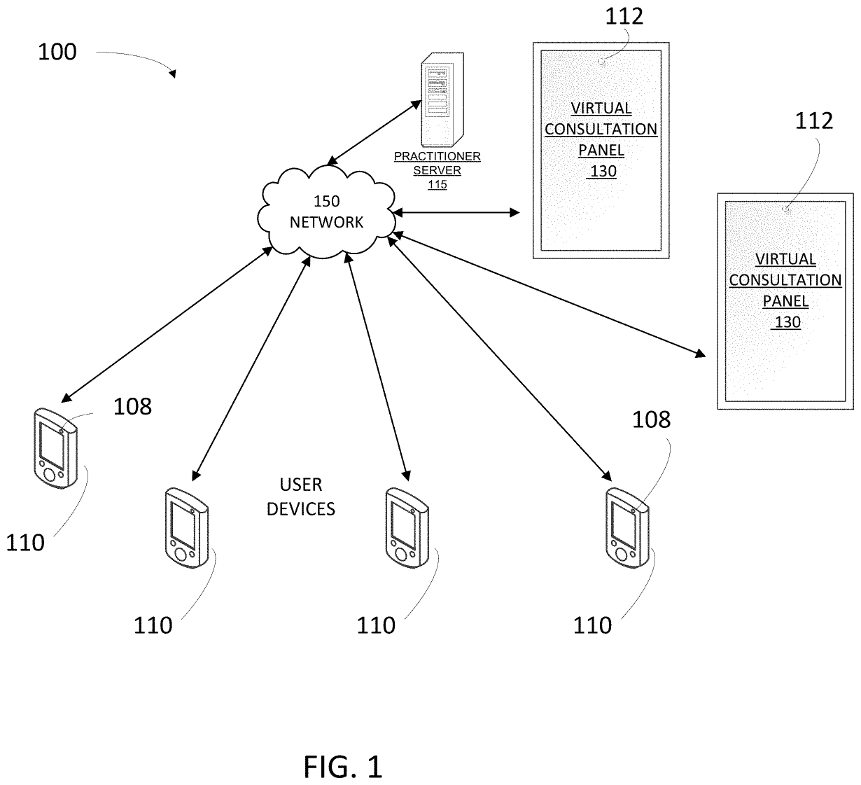

FIG. 1 illustrates an example system 100 for virtual consultations. As indicated in FIG. 1, system 100 can include one or more user devices 110, one or more virtual consultation panels 130, and one or more servers such as practitioner server 115, communicatively coupled via a network 150.

Network 150 can include, for example, any one or more of a local area network (LAN), a wide area network (WAN), the Internet, and the like. Further, the network 150 can include, but is not limited to, any one or more of the following network topologies, including a bus network, a star network, a ring network, a mesh network, a star-bus network, tree or hierarchical network, and the like.

User devices 110 may be implemented as a desktop computer, a laptop computer, or a tablet computer, a smartphone (e.g., an iPhone X®), a personal digital assistant (PDA), a mobile audio player, a Global Positioning System (GPS) receiver, a video game console, and/or any other personal computing device having a camera and communications circuitry for transmitting images to virtual consultation panel 130.

User devices 110 can be operated by a patient desiring a surgical consultation in a location of their choosing (e.g., in their own home). Virtual consultation panels 130 may be located in a doctor's office, remote from the user's location.

User devices 110 may each include a sensor 108 that includes at least one camera for capturing images. Sensors 108 may include, for example, a front-facing camera and/or a rear-facing camera. A user of user device 110 can operate one or more cameras of sensor 108 to capture, for example, a live video stream including images of some or all of the user's body. The user device 110 transmits the images of some or all of the user's body, via network 150, to one or more of virtual consultation panels 130.

Virtual consultation panels 130 include communications circuitry (not explicitly shown in FIG. 1) that receives the live video stream including the images from the user device 110, and displays the live video stream on a display panel of the virtual consultation panel. Virtual consultation panels 130 can also include cameras 112, microphones, or other components for receiving images, video, or audio of the consulting surgeon that can be transmitted to user device 110 to create a fully interactive virtual consultation. The display panel of the virtual consultation panel may have a size that is sufficient to display a life-size representation of some or all of the user's body in the images.

The outer surface of the display panel may be a mirrored surface. In some implementations, the entire outer appearance of virtual consultation panel 130 may mimic that of a full-length mirror. The virtual consultation panel 130 may be provided with a stand to be freestanding in a room, or can be a wall-mounted or wall-integrated device (e.g., a device that is partially embedded in a wall or other structure, or a device having an outer surface that is flush with the wall in which it is embedded).

### Example Virtual Consultation Panel

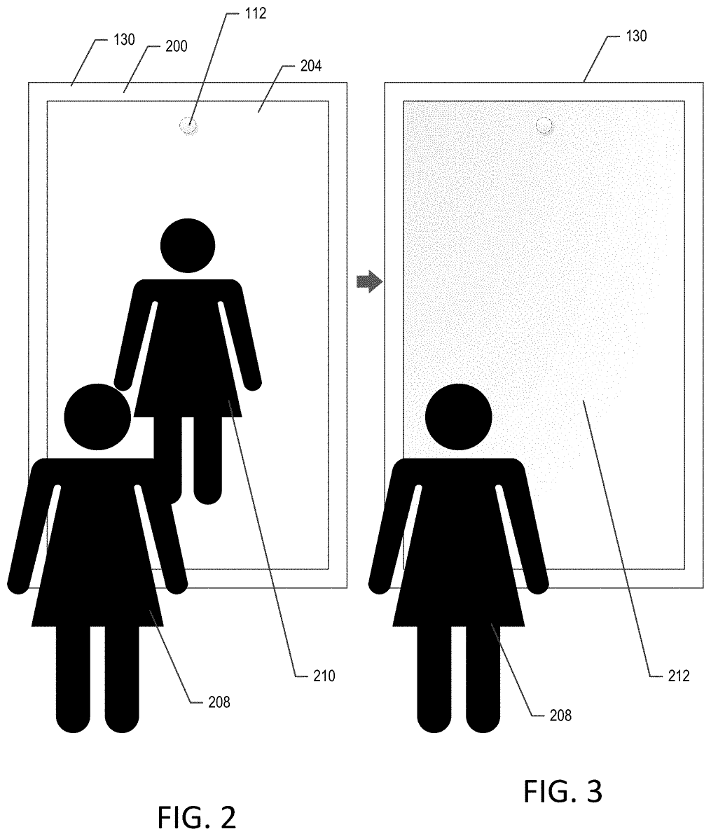

For example, FIGS. 2 and 3 illustrate an example in which the display panel of virtual consultation panel 130 is large enough to display an actual-size representation of the entire body of the user of a user device 110 (e.g., using a live video feed from the remote user's device), so that the body of the user (e.g., a patient) can be viewed and assessed by a consulting surgeon that is viewing the virtual consultation panel 130.

As indicated in FIG. 2, virtual consultation panel 130 may include a frame 200 and a mirrored outer surface 204. In this example, the display panel of virtual consultation panel 130 is inactive, and a reflection 210 of a consulting surgeon 208 is visible on the mirrored outer surface 204. In contrast, in the example of FIG. 3, display panel 212 is active, transmitting display light through mirrored outer surface 204 such that the reflection 210 is no longer visible.

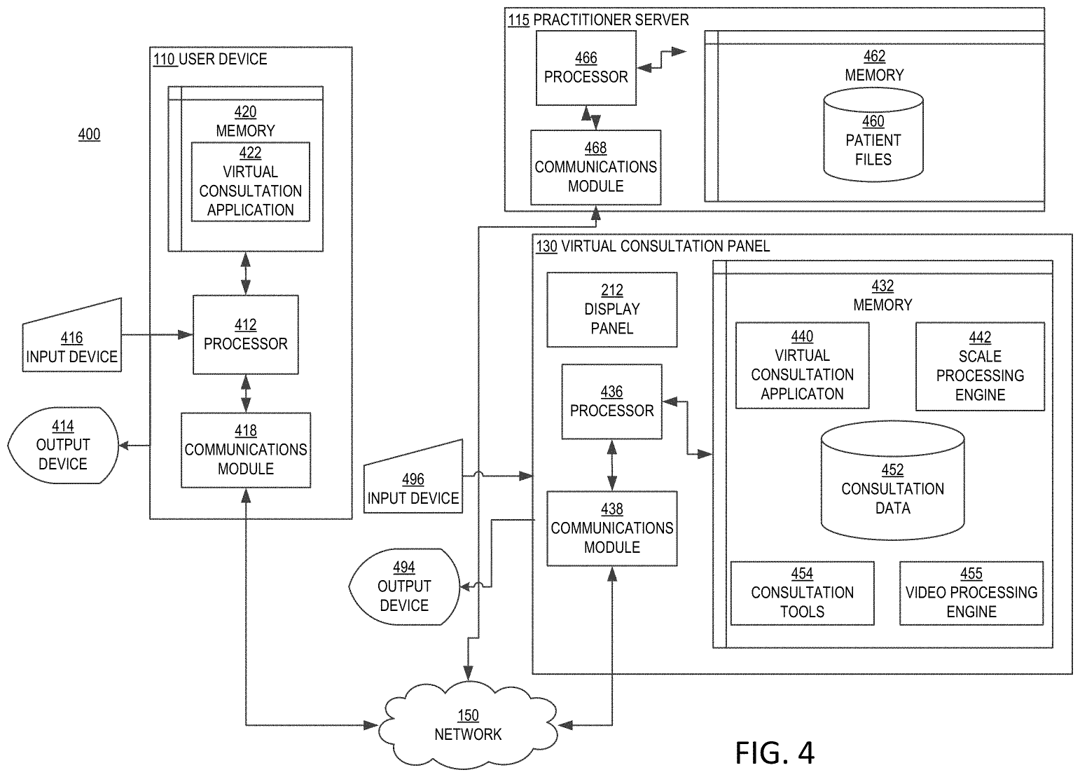

FIG. 4 is a block diagram 400 illustrating an example virtual consultation panel 130, practitioner server 115, and user device 110 in the system 100 of FIG. 1, according to certain aspects of the disclosure. The user device 110, the practitioner server 115, and the virtual consultation panel 130 are connected over the network 150 via respective communications modules 418, 468, and 438. The communications modules 418, 468, and 438 are configured to interface with the network 150 to send and receive information, such as data, requests, responses, video frames, scale information, and commands to other devices on the network. The communications modules 418, 468, and 438 can be, for example, modems or Ethernet cards.

The virtual consultation panel 130 includes a memory 432, a processor 436, and a communications module 438. The memory 432 of the virtual consultation panel 130 includes a virtual consultation application 440, a scale processing engine 442, and a video processing engine 455.

Virtual consultation application 440, when executed using processor 436, may provide a surgeon interface that manages the display of video from user device 110. Virtual consultation application 440 may also receive control input from an input device 496 such as a touch screen of display 212, or another input device, provide access to one or more consultation tools 454 stored in memory 432, and/or control the size of displayed video frames and/or the size of other features or scale indicators, using scale information received from user device 110 and processed by scale processing engine 442.

Scale processing engine 442 may receive scale information from user device 110. The scale information may be received as part of the content of the video frames in a live video stream from user device 110, and/or may include scale information provided as metadata or separate data along with the video stream. The scale information may include information indicating the size of one or more features of a user in the video frames, and/or information indicating optical features of a camera of the user device. Scale processing engine 442 may also obtain information associated with display panel 212 (e.g., the physical size and pitch of display pixels of the display panel). Scale processing engine 442 processes the received scale information and/or the information associated with display panel 212 to generate scaled images of the user for display with the display panel, to generate scale indicators for overlay on unscaled or scaled images of the user, and/or may provide processed scale information to virtual consultation application 440 to generate the scaled images (e.g., life-size or actual-size images) and/or the scale indicators for display. In this way, processor 436 scales the live video stream from user device 110 to generate a virtual consultation view of the live video stream, for display by the display panel in the active mode of the display panel.

Consultation tools 454 that may be provided to a consulting surgeon by virtual consultation application 440 may include tools for annotating, storing, cropping, or otherwise manipulating displayed images of a patient from user device 110, tools for adding audio, visual, or written notes to a patient file (e.g., locally in a consultation data storage 452 in the memory 432 of the virtual consultation panel, and/or remotely in patient file 460 in memory 462 of practitioner server 115 having a processor 466), tools for manipulating three-dimensional displays associated with the patient, or the like.

Video processing engine 455 processes the live video stream from user device 110 for display by virtual consultation application 440 (e.g., to scale the video stream using scale information received from scale processing engine 442 and/or to prepare the live video stream for modification by scale processing engine 442 and/or virtual consultation application 440).

The processor 436 of the virtual consultation panel 130 is configured to execute instructions, such as instructions physically coded into the processor 436, instructions received from software in memory 432, or a combination of both. For example, the processor 436 of the virtual consultation panel 130 executes instructions to receive a live video stream from user device 110, to receive scale information from user device 110, and to generate an actual-size image of a patient in the video stream for display by display panel 212.

Input device 496 of virtual consultation panel 130 may include a mouse, a handheld controller such as a virtual reality (VR) glove of other grasping controller, a television controller, a physical or virtual keyboard, or the like. Output device 494 may include one or more speakers, or one or more haptic components that provide a tactile response through or with display panel 212.

As shown in FIG. 4, memory 420 of user device 110 may also store a virtual consultation application 422 (e.g., a patient virtual consultation application) that, when executed by processor 412, provides a user interface for a patient (e.g., to display a live video feed of a consulting surgeon, and/or to provide one or more visual indicators of actions to perform for a virtual consultation). Virtual consultation application 422 may communicate with virtual consultation application 440 of virtual consultation panel 130 to execute a virtual consultation session between the patient and the consulting surgeon.

Virtual consultation application 422 may exchange automatic (e.g., background) communications with the virtual consultation application 440 of virtual consultation panel 130 to coordinate generation of reminders and selectable confirmation options and/or connection requests in advance of the virtual consultation session.

Input device 416 of user device 110 may include sensor 108 as described herein, which may be implemented to include a camera, a microphone, and/or one or more distance sensors, depth sensors, or other three-dimensional sensors that can obtain scale information to be provided by virtual consultation application 422 to virtual consultation application 440 at the virtual consultation panel. Output device 414 of user device 110 may include a display panel, a speaker, and/or tactile feedback components.

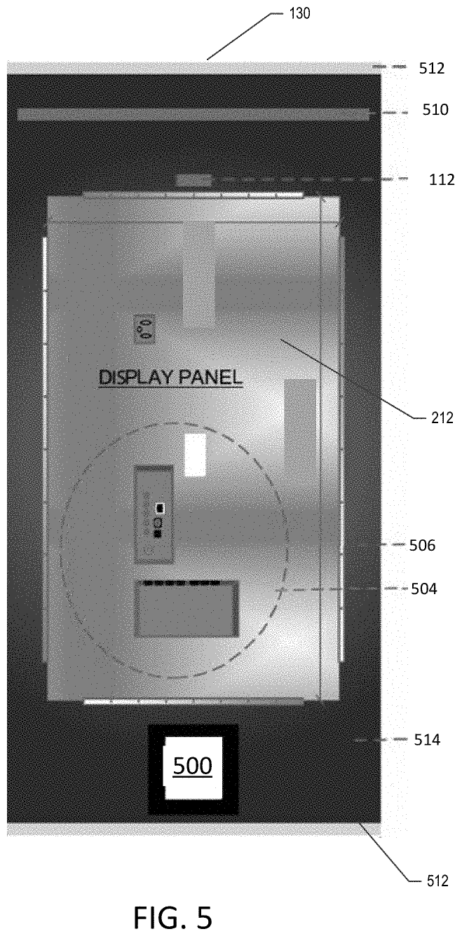

A rear view of virtual consultation panel 130 is shown in FIG. 5, illustrating one suitable implementation. In the example of FIG. 5, display panel 212 is mounted to a rear surface of a substrate 514. For example, substrate 514 may be a one-way mirror having a mirrored surface corresponding to the mirrored outer surface 204 of virtual consultation panel 130, and having a rear surface that reflects light that is incident through the mirrored outer surface, unless light such as display light from display panel 212 is projected outward from the rear surface through the mirrored outer surface.

As shown in FIG. 5, one or more additional components of virtual consultation panel 130 can be mounted to the rear surface of substrate 514. For example, a computing hub 500 and camera 112 may be mounted to the rear surface of substrate 514. In this configuration, camera 112 is coupled to the rear side of the substrate 514 and configured to capture a live video stream, using light that passes through the substrate 514 to the camera (e.g., an additional live video stream of a practitioner performing a surgical consultation using the scaled live video stream of the patient on display panel 212).

Computing hub 500 includes computing components for virtual consultation panel 130. The computing components can include one or more processors such as processor 436, one or more memories such as memory 432, communications circuitry such as communications module 438 for communications via network 150 (see FIG. 1), video processing circuitry such as video processing engine 455 for processing video streams from user devices for display on display panel 212, video processing circuitry such as video processing engine 455 for processing images and/or video from camera 112 and providing associated images and/or video for transmission to one or more user devices, and/or scale processing circuitry such as scale processing engine 442. The computing components can include input interfaces for receiving input from an input device 496 such as a touch-sensitive surface of display panel 212 and/or substrate 514, from a mouse, from a handheld controller such as a virtual reality (VR) glove or other grasping controller, from a physical or virtual keyboard, or the like. The computing components can include output interfaces for outputting video and/or audio data to input/output interfaces 504 of display panel 212, output interfaces for outputting video and/or audio data to communications module 438 for transmission to user device 110 and/or practitioner server 115, and/or output interfaces for outputting data to output device 494.

Input/output interfaces 504 may include optical connectors, coaxial connectors, high-definition media input (HDMI) connectors, universal serial bus (USB) connectors, serial connectors (e.g., DVI connectors or VGA connectors), s-video connectors, composite connectors, electrical power connectors, Ethernet connectors, ex link connectors, and/or any other connectors or other interfaces for receiving electrical and/or communications signals from computing hub 500, external network components, or the like.

The computing components can include memory and/or storage such as memory 432 for storing consultation information (e.g., consultation data 452) generated during a virtual consultation operation with virtual consultation panel 130. Consulting information stored as consultation data 452 can include captured still images from a patient video stream, video clips from a patient video stream, image annotations input to virtual consultation panel 130, practitioner video notes, practitioner audio notes, patient size information, and/or other information generated by operation of virtual consultation panel 130 during a virtual consultation. The computing components of computing hub 500 (e.g., communications module 438) can also be used to transmit the consultation information to the practitioner server.

Camera 112 is arranged, in the example of FIG. 5, to capture images of consulting surgeon 208 through substrate 514 (e.g., using light that passes through mirrored outer surface 204 onto camera 112). For example, camera 112, or one or more lenses thereof, may be mounted behind one or more corresponding non-mirrored portions of virtual consultation panel 130. Camera 112 can be operated to capture video and/or audio (e.g., in an additional live video stream) of the consulting surgeon for transmission to the user device 110 of a patient.

FIG. 5 also shows how one or more mounting brackets 506 may be provided that attach display panel 212 to substrate 514. In this example, four mounting brackets 506 are provided that attach the right, left, top, and bottom edges of display panel 212 to substrate 514. However, it should be appreciated that more or fewer than four mounting brackets may be used, and/or other attachment mechanisms (e.g., adhesives or other bonding materials interposed between interfacing surfaces of display panel 212 and substrate 514) can be used to attach display panel 212 to substrate 514.

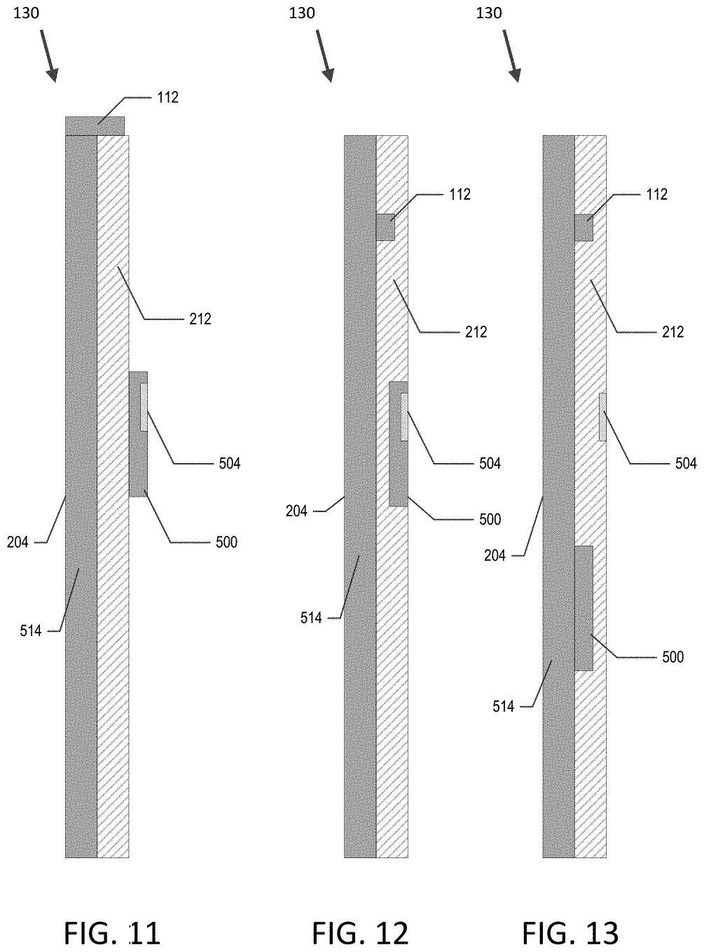

However, it should be appreciated that these examples, in which display panel 212 is manufactured separately from substrate 514 and later mechanically attached to a separate substrate, are merely illustrative. In other some implementations (see, e.g., FIGS. 11-13 below), the components of display panel 212, computing hub 500, and/or camera 112 can be built directly on a common substrate (e.g., with the mirrored outer surface of virtual consultation panel 130 being the outermost surface of display panel 212).

Returning to the example of FIG. 5, mounting structures 510, and rear portions 512 of frame 200 can also be seen. Mounting structure 510 may be, for example, a bracket such as a hangman bracket that is attached to the rear surface of substrate 514 or display panel 212 for hanging or otherwise mounting virtual consultation panel 130 (e.g., in a wall-mount implementation) on another structure. However, it should be appreciated that mounting structure 510 is merely an example of a structural support member that can be provided to support substrate 514 and display panel 212 for viewing of the display panel. In the example of FIG. 5, rear portions 512 of frame 200 may be rear portions of a j-channel frame that extends from rear portions 512 over the edges of substrate 514 to form the front portions of frame 200 shown in FIGS. 2 and 3.

In the example rear view of FIG. 5, display panel 212 is substantially smaller in area than substrate 514. In this arrangement, when display panel 212 is operating (e.g., generating display light that is emitted through substrate 514 for viewing by the consulting surgeon), the active area of the display panel will be surrounded by mirrored portions of mirrored outer surface 204, which may be surrounded by frame 200. However, it should be appreciated that, in other arrangements (see, e.g., FIGS. 11-13 below), the active area of the display panel can extend to the edge of substrate 514 and mirrored outer surface 204 of the substrate (e.g., to frame 200 or to a frameless edge of mirrored outer surface 204).

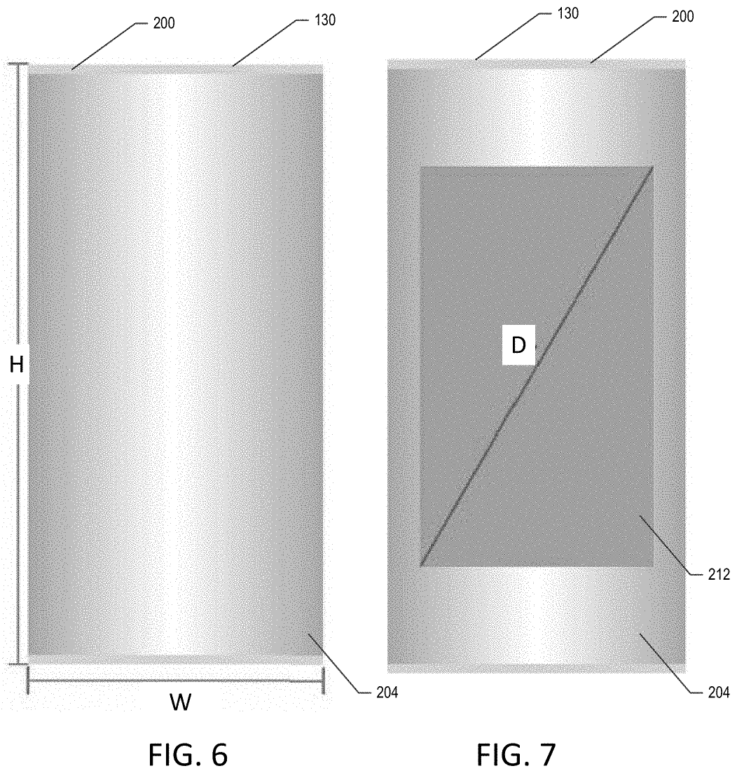

FIGS. 6 and 7 illustrate front views of virtual consultation panel 130 in the configuration of FIG. 5. As indicated in FIG. 6, in the example of FIGS. 5-7, the front surface 204 of virtual consultation panel 130 may have a height, H, and a width, W. The height, H, of the front surface 204 may be, for example, between three feet and eight feet (e.g., 66 inches). The width, W, of the front surface 204 may be, for example, between eighteen inches and six feet (e.g., 32 inches).

In the example of FIG. 6, display panel 212 is inactive (e.g., in an inactive display mode for the display pane, as in the example of FIG. 2). In the example of FIG. 7, display panel 212 has been activated (e.g., in an active display mode for the display panel, as in the example of FIG. 3). FIG. 7 shows how, in the configuration of FIGS. 5-7, display panel 212 has a display area that is smaller than the surface area of outer surface 204 of virtual consultation panel 130. For example, the display panel 212 in the configuration of FIG. 7 has a diagonal width, D, which may be between 12 and 96 inches (e.g., 49 inches). However, as discussed elsewhere herein, the example configuration of FIGS. 5-7 in which display panel 212 has a display area that is smaller than the overall outer surface of the virtual consultation panel 130 is merely illustrative, and other configurations are contemplated.

More generally, each or either of display panel 212 and/or substrate 514 may have an outer surface with a height of between three feet and eight feet and a width of between eighteen inches and six feet. In this way, a virtual consultation panel 130 is provided that is sufficiently large to display actual-size (or nearly actual-size) representations of the entire patient, or at least the portion of the patient that is being considered for surgery (e.g., the patient's torso, stomach, arm, leg, breast, or a portion thereof) in a virtual consultation view of the live video stream.

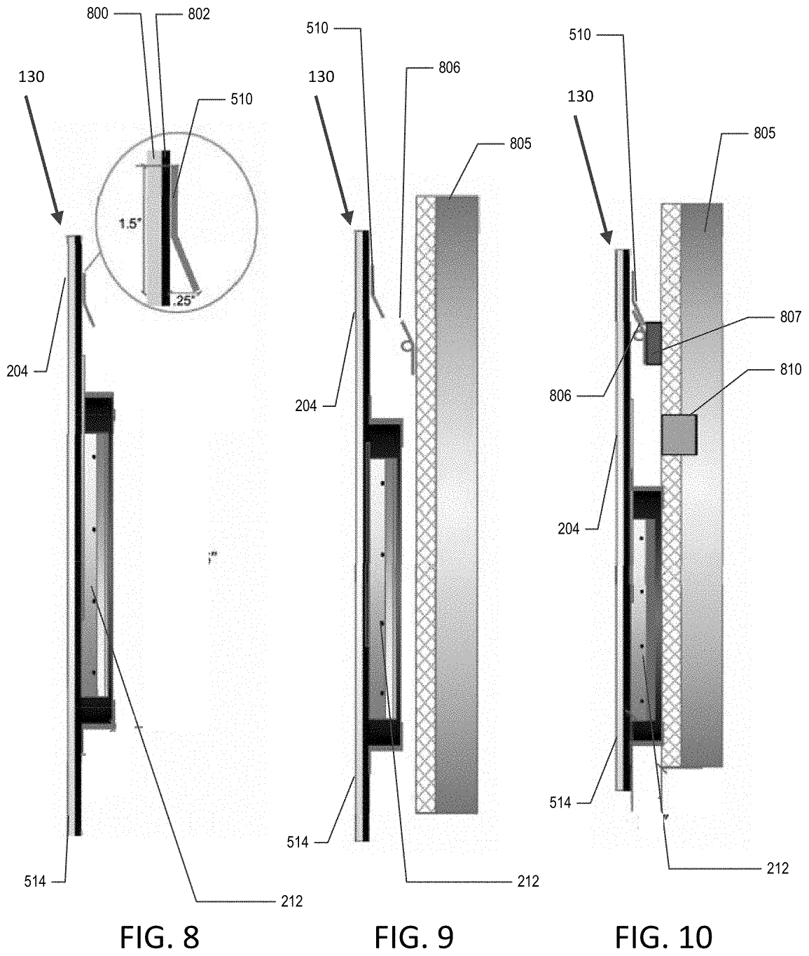

FIGS. 8-10 show side views of the virtual consultation panel of FIGS. 5-7, to illustrate various aspects of a mounting operation for a wall-mounted configuration for the panel. For example, as shown in FIG. 8, mounting structure 510 may include an upper portion that is attached to the rear surface of substrate 514, and an extending portion that angularly extends away from the rear surface.

FIG. 8 also illustrates a dual-layer configuration for substrate 514, in which substrate 514 includes a front layer 800 and a rear layer 802. For example, front layer 800 may be a glass outer layer that forms the outer surface 204 of virtual consultation panel 130. Front layer 800 may be attached to and/or coated with a rear layer 802, which may be formed from (as examples) metal and/or another reflective substrate or coating, a light-absorbing paint or other light-absorbing coating or substrate, and/or any other suitable substrate or coating that causes light that is not reflected from the outer surface of transparent front layer 800 and passes through front layer 800 to rear layer 802 to be reflected or absorbed without passing through rear layer 802. Although a dual-layer substrate 514 is illustrated in FIG. 8, other configurations (e.g., a single layer reflective substrate and/or a substrate having more than two layers) are contemplated.

In the example of FIG. 9, an interfacing bracket 806 is attached to a support structure 805 such as a wall in an office of a medical practitioner, such as a surgeon. In this configuration, mounting structure 510 on virtual consultation panel 130 interfaces with interfacing bracket 806 to mount virtual consultation panel 130 to support structure 805. In the example of FIG. 9, display panel 212 extends outwardly from the rear surface of substrate 514 beyond the extent of mounting structure 510. In this configuration, support structure 805 may be provided with a recess (not shown) into which the extending portion of display panel 212 is received (e.g., in a wall-embedded implementation of virtual consultation panel 130), to allow the rear surface of substrate 514 to lie flush, or nearly flush with the interfacing surface of support structure 805.

FIG. 10 illustrates a wall-mounted configuration for virtual consultation panel 130, in which interfacing bracket 806 is attached to a protruding structure 807 on support structure 805. Protruding structure 807 may be an integral protrusion of support structure 805, or may be a separate extending member that is attached to support structure 805 and interposed between support structure 805 and interfacing bracket 806. In the example of FIG. 10, an interface 810 for providing electrical and/or communication signals to and/or from virtual consultation panel 130 is also embedded within support structure 805. Interface 810 may include connectors of cables for coupling to any or all of input/output interfaces 504 of FIG. 5.

As described above, configurations other than the configurations of display panel 212 and substrate 514 of FIGS. 5-10 are contemplated herein. For example, FIG. 11 illustrates a side view of virtual consultation panel 130 in a configuration in which display panel 212 is formed directly on the rear surface of substrate 514 (e.g., a glass or other transparent substrate). In this configuration, display panel 212 (e.g., including the array of display pixels that generate the display light that travels through substrate 514 to display images of a remote patient) extends to the upper and lower edges (e.g., and the left and right edges though not visible in FIG. 11) of substrate 514. In this configuration, the entire outer surface of virtual consultation panel 130 is mirrored when display panel 212 is inactive, and the entire outer surface of virtual consultation panel 130 projects display light when display panel 212 is active.

In can be seen in FIG. 11 that computing hub 500 can be attached to the rear surface of display panel 212 (e.g., for direct interface to the circuitry of display panel 212) with input/output components 504 thereon. In this example, camera 112 is mounted to a top surface of virtual consultation panel 130. However, this is merely illustrative. For example, FIGS. 12 and 13 illustrate configurations in which camera 112 is integrally formed on the interior surface of substrate 514 (e.g., embedded within and/or surrounded by display panel 212) to receive light through substrate 514 for capturing video of a consulting surgeon.

FIGS. 12 and 13 also illustrate, respectively, implementations in which computing hub 500 is integrally formed with display panel 212 (e.g., formed by one or more circuit boards and/or integrated circuits disposed within a housing of display panel 212) and in which computing hub is formed on substrate 514 (e.g., by one or more circuit boards and/or integrated circuits formed by or on the substrate or attached to the substrate 514). In the example of FIG. 12, input/output interfaces 504 are formed on a rear surface of computing hub 500 that is flush or nearly flush with the rear surface of display panel 212. In the example of FIG. 13, input/output interfaces 504 are disposed at the rear surface of display panel 212 and separate from computing hub 500.

In the configurations of FIGS. 11, 12, and 13, substrate 514 may be a single layer, dual layer, or other multi-layer substrate as described herein, and/or portions of display panel 212 (e.g., the display pixels themselves) can form the light absorbing inner surface of virtual consultation panel 130 that causes substrate 514 to have a mirrored outer surface 204 when display panel 212 is inactive. In the configurations of FIGS. 11, 12, and 13, virtual consultation panel 130 is shown without a frame 200 or mounting structures, for clarity. However, any of the configurations of FIG. 11, 12, or 13 can be provided with a frame 200 and/or mounting structures such as mounting structure 510 (e.g., for a wall-mounted or wall embedded implementation), and/or a stand for a free-standing implementation of virtual consultation panel 130.

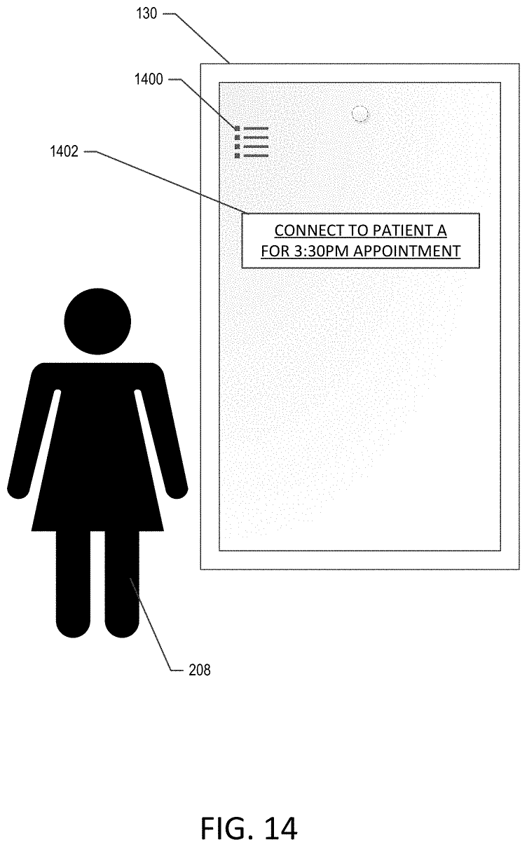

Turning now to FIG. 14, virtual consultation panel 130 is shown in a configuration in which the virtual consultation panel 130 has received a request for connection from one of user devices 110. For example, virtual consultation panel 130 may send instructions to one of user devices 110 that is associated with an imminent appointment (e.g., an appointment scheduled to begin in less than five minutes, less than ten minutes, less than fifteen minutes, or less than thirty minutes) to request a connection to the virtual consultation panel 130. Responsively, the user device 110 may provide a selectable option to the patient to request the connection. In this way, when the patient selects the selectable option to request the connection, virtual consultation panel 130 is informed by user device 110 that the patient is ready for the imminent consultation (scheduled for patient “A” at 3:30 pm in the example of FIG. 14).

Responsive to receiving the request, display panel 212 is operated by processor 436 to display selectable connection option 1402, including patient and scheduling information for the imminent appointment. Consulting surgeon 208 can activate the virtual consulting session by selecting the selectable connection option 1402. The selectable connection option 1402 can be selected by touching the display panel within the boundaries of the selectable connection option 1402 (e.g., in configurations in which the display panel is touch-sensitive), or using a mouse, a keyboard, a remote control, or other controller for virtual consultation panel 130 to click, tap, or otherwise select option 1402.

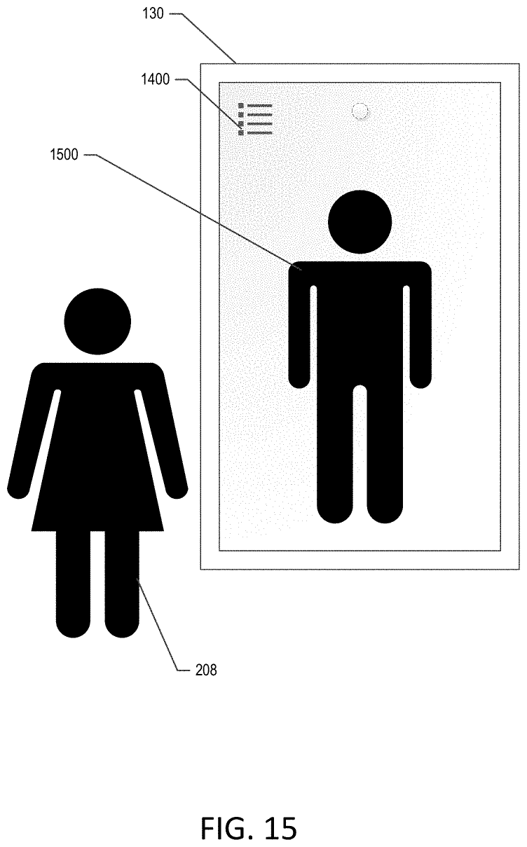

When the selectable connection option 1402 is selected, a one-way or two-way video conferencing session is established between virtual consultation panel 130 and user device 110 of patient A. Once the video conferencing session has been established, display panel 212 displays a live video feed from the user device, including images of the patient, such as images of a patient 1500 as illustrated in FIG. 15. As can be seen in the example of FIG. 15, display panel 212 and virtual consultation panel 130 are sufficiently large that a life-sized image of the patient 1500 can be displayed for viewing and consultation by consulting surgeon 208. In other configurations, the virtual consultation panel 130 can be provided with a display panel sized to display actual or life-sized images of a particular portion of the patient's body (e.g., the abdomen, torso, arm, leg, breast, etc.) being considered for a surgery.

In the example of FIG. 15 in which practitioner 208 is shown performing a surgical consultation using the displayed live video stream of patient 1500, when the patient 1500 moves and speaks, the consulting surgeon is provided with real-time video and audio of the user's movement and speech. In this example, camera 112 of virtual consultation panel 130 also captures video and audio from the consulting surgeon, and virtual consultation panel 130 transmits that video and audio, in real time, to the user device 110 of the patient. In this way, a two-way video session between patient 1500 and consulting surgeon 208 is provided. In circumstances in which the communications bandwidth available to the patient is limited, a one-way video session may be provided in which the patient only receives audio from virtual consultation panel 130.

As indicated in FIGS. 14 and 15, in operation, a virtual control element such as a selectable menu option 1400, can be displayed. Selectable menu option 1400 allows the consulting surgeon to select display options for how the user is displayed on the display panel, and/or to access other functions of the virtual consultation panel (e.g., using a remote controller, an in-panel controller, or touch-sensitive interface of the display panel). A touchscreen capability of display panel 212 may also allow the consulting surgeon to move, alter, or manipulate the display of the representation of the user (e.g., to zoom in or out, rotate, brighten, darken, annotate, add contrast, freeze, capture a still image, etc.). As described in further detail hereinafter, virtual consultation panel 130 can also store and/or transmit for remote storage at a server, images, or other consultation information generated by virtual consultation panel 130.

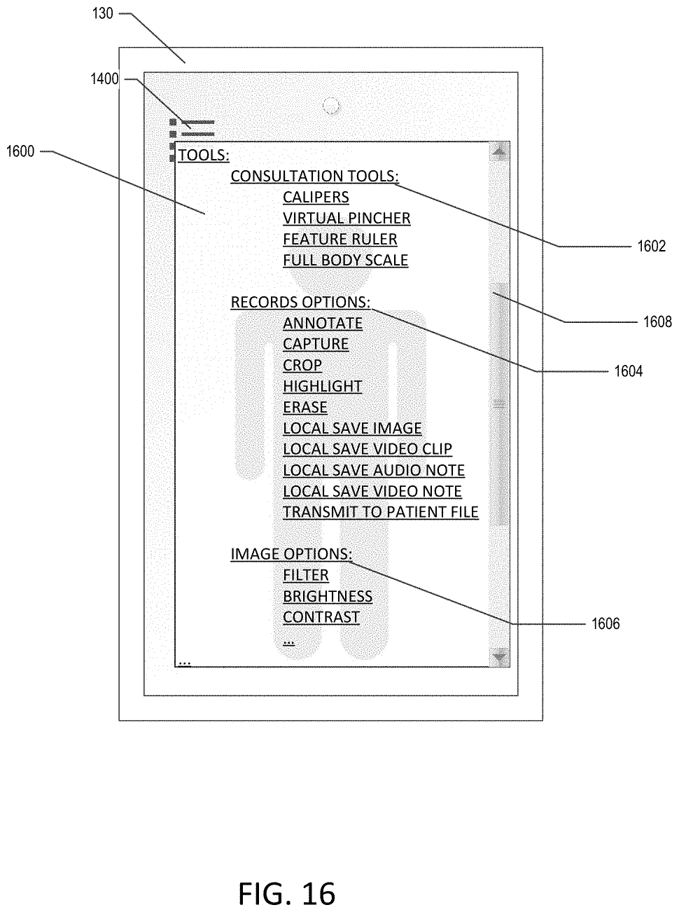

Selectable menu option 1400 can be displayed by display panel 212. Selecting the selectable menu option 1400 causes one or more selectable menu items to be displayed by display panel 212. FIG. 16 shows examples of selectable menu items that may be provided by virtual consultation panel 130.

In the example of FIG. 16, menu option 1400 has been selected, and virtual consultation panel 130 is displaying a tools menu 1600, including a plurality of selectable tools, such as consultation tools 454, for a virtual surgical consultation using the live video stream from user device 110. In the example of FIG. 16, the selectable tools include consultation tools 1602, records options 1604, and image options 1606. In this example, tools menu 1600 is scrollable using scrollbar 1608. Consultation tools 1602, records options 1604, and image options 1606 of tools menu 1600 may be links to corresponding tools in consultation tools 454 stored in memory 432 of virtual consultation panel 130.

Consultation tools 1602 may be selected for use by consulting surgeon 208 during a virtual consultation. As shown in FIG. 16, the consultation tools 1602 may include a virtual calipers, a virtual pincher, a virtual feature ruler, or a virtual full body scale. Each of these consultation tools, when selected, can be displayed as an overlay on the images of patient 1500 on display panel 212. For example, the virtual feature ruler can be generated based on scale information provided from user device 110 and placed over a particular feature of the image of patient 1500 to allow the consulting surgeon to determine the size of that feature.

The virtual feature ruler can be automatically placed by virtual consultation panel 130 (e.g., by detecting the desired feature for a particular consultation in the images of the patient) or can be dragged to, and oriented over the feature by the consulting surgeon. The virtual body scale can be displayed along an edge of the display panel to allow the consulting surgeon to determine the height and/or overall size of the patient. The virtual calipers may be an expandable or contractible ruler that displays the size of an indicated region in an image to allow the consulting surgeon to measure particular feature sizes in the image of the patient. For example, the virtual calipers may be manipulable via input to the display panel 212 to measure one or more features of the body of the patient. The virtual pincher may be a tool that allows the consulting surgeon to virtually pinch a portion of the user's body. Based on sensor information from the user device (e.g., three-dimensional size and/or other biometric information), the virtual pinch input to virtual consultation panel 130 may cause the processor of virtual consultation panel 130 to deform that pinched portion of the image of patient 1500 as the actual body of the patient would deform on a physical pinch.

In the example of FIG. 16, records options 1604 that may be provided by virtual consultation panel 130 may include options to annotate the image of the patient, capture a still image of the patient from the live video feed being displayed, crop the video feed or the captured still image, highlight a portion of the video feed or the captured still image, erase an annotation, a highlight, or a portion of the video feed or the still image, save the still image in local memory 432 of virtual consultation panel 130, save a video clip from the live video stream in local memory 432 of virtual consultation panel 130, save an audio note (e.g., a spoken note from the consulting surgeon recorded using a microphone of virtual consultation panel 130) in local memory 432 of virtual consultation panel 130, save a video note (e.g., a video recorded using camera 112 of virtual consultation panel 130) in local memory 432 of virtual consultation panel 130, and/or to transmit any of the above to a patient file, such as a remote patient file 460 on practitioner server 115.

For example, while the patient is pinching a portion of their body, the consulting surgeon may use an annotation tool to draw on the portion of the patient in the video images, and then store that annotated portion of the video stream locally in the memory 432 of virtual consultation panel 130, and/or remotely at practitioner server 115 for later reference (e.g., in preparation for a later surgery for that patient).

FIG. 16 also shows how image options 1606 can include options to filter the video stream and/or a still image from the video stream, and/or change the brightness, contrast, or other features of the video stream and/or still image to allow the consulting surgeon to better view one or more portions of the video stream or the still image.

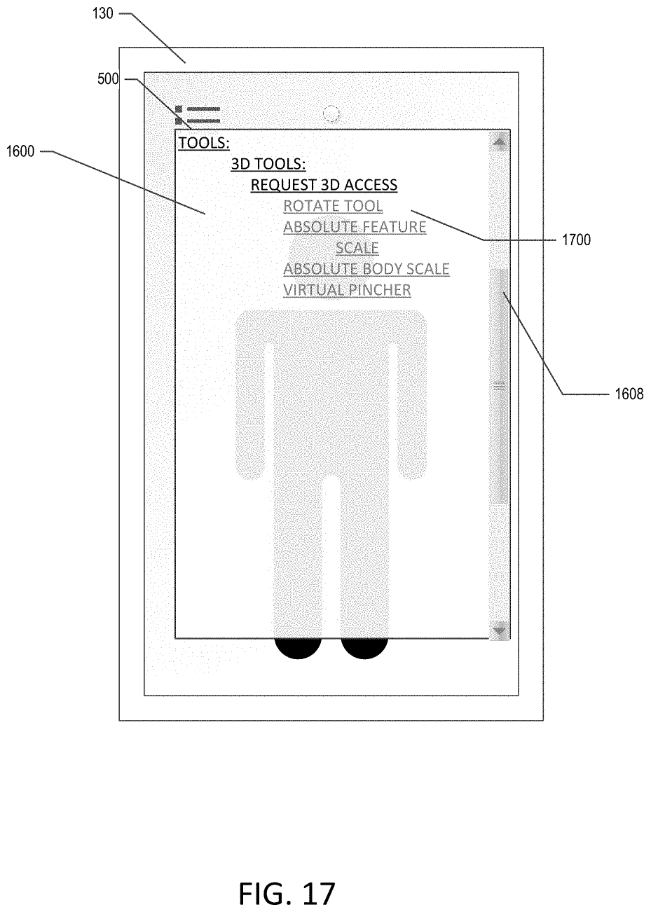

FIG. 17 illustrates additional tools that may be provided by virtual consultation panel 130 in tools menu 1600. In the example of FIG. 17, three-dimensional (3D) tools menu 1700 is shown. In this example, a first 3D tool for requesting access to 3D information from the patient's device is provided, with other unselectable options that become selectable when 3D access is provided by the patient device. For example, the consulting surgeon may select the request 3D access option from menu 1700, to cause virtual consultation panel 130 to request 3D or other scale information from user device 110. The 3D tools in three-dimensional (3D) tools menu 1700 may be links to corresponding tools in consultation tools 454 stored in memory 432 of virtual consultation panel 130.

If the 3D access is granted (e.g., automatically by the user device, or by express permission input to the user device by the patient), 3D sensors, depth sensors, and/or other scale sensors of sensor 108 of user device 110 are activated. Sensor 108 then provides a three-dimensional model of the portion of the patient in the image and/or a depth map corresponding to the displayed image of the patient. Based on this received 3D/scale information, tools such as a rotate tool, an absolute feature scale tool, an absolute body scale tool, and/or a virtual pincher may be provided. The feature scale, body scale, and pincher of FIG. 17 may correspond to the feature scale, the body scale, and the pincher of FIG. 16 in circumstances in which 3D information is automatically provided from user device 110. However, in other circumstances, the feature scale, the body scale, and the pincher of FIG. 16 may operate using scale information estimated by virtual consultation panel 130 (e.g., based on image features and/or known user device features) while the feature scale, body scale, and pincher of FIG. 17 are absolute-scale tools based on 3D measurement from sensor 108 of the user device.

In some circumstances, if a three-dimensional model of a portion of the user is provided by user device 110 to virtual consultation panel 130, the 3D model itself (and/or a combination of the 3D model and corresponding images of the patient) may be displayed on display panel 212 as a 3D representation of the patient. In these circumstances, the rotate tool may allow the consulting surgeon to virtually rotate and/or otherwise manipulate the 3D virtual representation of the patient displayed on the display panel. The virtual pincher in these circumstances may show a virtual pinch of the 3D representation on the display panel (e.g., with or without tactile feedback simulating the pinch to the consulting surgeon such as through the display panel or with haptic components of a VR glove or other controller).

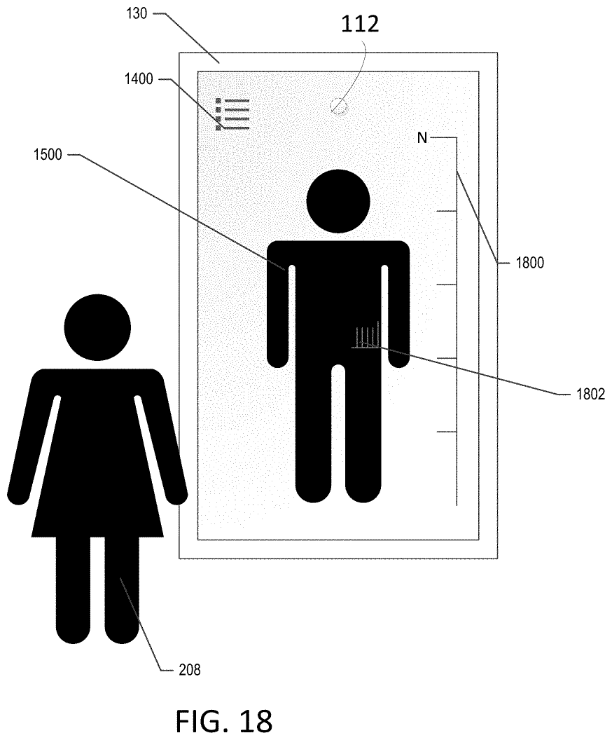

FIG. 18 illustrates examples of an absolute body scale (e.g., a virtual patient scale 1800) and an absolute feature scale (e.g., virtual feature scale 1802) that can be displayed on the display panel 212. One or both of virtual patient scale 1800 and virtual feature scale 1802 can be scaled, by virtual consultation panel 130, to the absolute scale of the image of the user, as described above. For example, if the virtual feature scale 1802 has an overall length of five inches, as displayed on the display panel 212, the size of the portion of the image of patient 1500 over which the virtual feature scale 1802 is overlaid will correspond to a five-inch portion of the patient's body at the patient's remote location. Virtual consultation panel 130 obtains the absolute scale information and the images in the video stream from user device 110, determines, based on a physical size of the pixels of the display panel 212, the absolute scale information, and the pixel size in the images in the video stream, the size of the images to be displayed on display panel 212, and the size of the virtual feature scale 1802, to ensure the size correspondence. In this way, the consulting surgeon 208 is able to perform a surgical consultation with a remote patient, as if that patient is in the room with the surgeon.

It should also be appreciated that, in some implementations, a permanent scale feature such as a ruler (e.g., a ruler indicating one or more lengths between one sixteenth of an inch to several feet, or lengths in other units) is attached to mirrored outer surface 204, engraved or otherwise embedded in mirrored outer surface 204, attached to frame 200, engraved or otherwise embedded in frame 200, or attached to frame 200. In these implementations, virtual consultation panel 130 may automatically display the images of patient 1500, scaled to the scale indicated by the ruler (e.g., based on three-dimensional depth and/or size information provided from a sensor of the user device, based on known camera features of the user's device, and/or based on a known pixel scale of display panel 212).

In general, one or more scale indicators (e.g., rulers) by which the consulting surgeon can gauge the actual physical size of the displayed user, or a particular portion of the user's body, such as a pinched portion of the user's body can be provided with virtual consultation panel 130. The scale indicators may be static indicators that are permanently included on or near the display panel (e.g., a scale indicator formed in a semi-transparent layer attached to the outer surface of the display panel, a scale indicator etched or printed on the outer surface or embedded within the mirror layer of the display panel, or a scale indicator printed on, embedded in, or attached to a frame of the virtual consultation panel 130), or may be virtual scale indicators that are generated and/or scaled when the display panel is operating (e.g., with a permanent static size, or with a size and/or position that is based on the images that are displayed).

It should also be appreciated that user device 110 of the patient may also be used to provide patient medical information (e.g., the patient's height, weight, medications, surgical history, and/or medical conditions or concerns that may be relevant to the consultation) to the virtual consultation panel 130. Virtual consultation panel 130 may temporarily store and/or display the patient medical information on the display panel 212 (e.g., along with or overlaid on the video stream from the user) to be considered by the surgeon.

In circumstances in which absolute scale information is not available from the user device sensors (e.g., in cases in which the patient has an older mobile phone), instructions may be provided by the consulting surgeon, or automatically generated by the virtual consultation panel, to take actions to allow virtual consultation panel 130 to determine an approximate size of the user in the images. For example, instructions may be provided from virtual consultation panel 130 to user device 110 to instruct the patient to stand or place a hand at a certain distance from the camera. Then, using a known or estimated height of the patient or size of the patient's hand, and based on the pixel distribution of patient or the hand in the images from the user device, an approximate size can be determined for patient and portions thereof, without 3D mapping, depth mapping, or other scale-determining sensors.

In some scenarios, the user can be provided with a physical measuring tool (e.g., by mail, courier, or electronic transmission of a printable tool) such as a ruler, a pincher or a caliper that can be placed on or near a part of the patient's body in a way that is visible to the consulting surgeon on the virtual consultation panel. Written instructions, or verbal instructions from the consulting surgeon can be provided via virtual consultation panel 130 and/or user device 110 for use of the provided tool(s) during consultation.

During the virtual consultation, instructions from the consulting surgeon and/or automatic instructions generated by virtual consultation panel 130 are conveyed from virtual consultation panel 130 to user device 110, and provided by user device 110 to the patient. For example, the virtual consultation panel 130 can be used to provide instructions to the user device 110 to instruct the patient to assume various positions and/or to perform various actions during the consultation.

For example, as part of the virtual consultation, virtual consultation panel 130 may provide instructions to the user device 110 to provide instructions to the patient to move into a front-facing position relative to the user device, a rear-facing position relative to the user device, a right-lateral-facing position relative to the user device, a perspective-facing position, and/or a left-lateral-facing position relative to the user device.

In some implementations, the instructions can include instructions to the user device 110 to display visual indicators of one or more of the front-facing position, the rear-facing position relative, the right-lateral-facing position, perspective-facing position, and/or the left-lateral-facing position.

For example, as described above, sensors 108 of the user device 110 of patient 1500 may include, in addition to a camera, one or more distance sensors or other sensors by which the user device can capture and/or transmit size and/or distance information and/or scale information associated with the patient in the images. In addition to allowing virtual consultation panel 130 to display the images of the user in actual size as described above (e.g., so that a consulting doctor such as a surgeon can be provided with a virtual consultation view of a video stream with which the doctor can assess the actual physical features of the user remotely), this distance information and/or scale information can be used by user device 110 (e.g., by a virtual consolation application running on the user device) to size one or more visual indicators for the patient.

For example, virtual consultation panel 130 can provide the instructions to the user device to display, using at least one depth sensor (e.g., an infrared sensor or other depth sensor in sensor 108) at the user device 110, one or more visual indicators of virtual consultation positions, with a displayed size that causes the patient to move to a particular distance from the user device to obtain an image of known size of the patient.

The instructions to the user device can also include instructions to display (e.g., using the at least one depth sensor at the user device 110) a correctness indicator for each of the front-facing position, the rear-facing position relative, the right-lateral-facing position, and the left-lateral-facing position. For example, visual indicators can change color, turn bold, or otherwise change or disappear when the user is in the desired position, at the desired distance.

During the consultation, instructions may be provided from virtual consultation panel 130 to user device 110 to provide video of a pinch of a part of their body. For example, instructions may be provided via virtual consultation panel 130 and user device 110 for the patient to pinch a portion of their stomach, side, arm, leg, or other body part in view of sensor 108 of the user device.

Instructions may be provided from virtual consultation panel 130 to user device 110 to provide scale information for the pinched at least part of at least the portion of the body of the user. The scale information can include depth, size, and/or scale information generated by user device 110 using sensor 108 (e.g., a three-dimensional model of the pinched portion as generated by user device 110 using sensor 108 or a depth map of the pinched portion as generated by user device 110 using sensor 108). However, in circumstances in which user device 110 does not include depth sensors, or in which depth sensor information is not available, instructions may be provided to the patient to perform other actions to provide the scale information.

For example, the patient may be instructed to place their hand at one or more distances from the camera of the user device. A virtual consultation application running on a user device, or a scale-determining engine at virtual consultation panel 130 may determine the size (e.g., a distance from thumb-tip to first finger-tip or from wrist to finger-tip) based on one or more images of the user's hand and the (e.g., approximately) known distance of the hand in the images. The size of the pinched portion can then be determined (e.g., by the virtual consultation application running on the user device or by the scale-determination engine at the virtual consultation panel 130) based on the images of the pinched portion and the hand pinching the portion, and the determined size of the patient's hand.

In other scenarios, the patient's hand and/or the pinched portion can be placed alongside a ruler or other scale-identifying tool (e.g., as provided to the patient by courier or as printed by the patient) so that the scale of the pinched portion can be determined from the video images.

Once the scale information is provided to virtual consultation panel 130, the virtual consultation panel (given the known pixel size of the display panel 212) can display an absolute-scale representation of the pinched portion of the body of the user for review by the consulting surgeon. Although some of these scale-determination operations (e.g., via imaging of the patient's hand) may only provide approximate scale information (e.g., in comparison with the highly accurate scale information provided by a sensor 108), the consulting surgeon can combine this approximate scale information with other medical information provided to virtual consultation panel 130 to determine the candidacy of the patient and various expectations for an upcoming surgery.

Particularly in cases in which the scale information provided from user device 110 to virtual consultation panel 130 includes information from a depth sensor of sensor 108 of the user device, the displayed pinched portion of the patient's body can be displayed in actual (life) size for the surgeon's review.

In cases in which the scale information includes a three-dimensional model of the patient or the pinched portion of the patient, the model may be used to display a three-dimensional view of some or all of the user's body on the display panel of the virtual consultation panel 130. This three-dimensional view may be a display of the model itself or can be a display of the images of the patient in the video stream with meta-data for the three-dimensional model. In this way, the view of the patient displayed on virtual consultation panel 130 can be rotated, pinched, or otherwise manipulated (e.g., via touch input to the panel) in three dimensions by the consulting surgeon.

As described above in connection with, for example, FIG. 4, user device 110 and virtual consultation panel 130 may exchange communications in preparation for an upcoming virtual consultation, for example, when an upcoming consultation has been detected by a virtual consultation application running on user device 110 and/or by virtual consultation panel 130. Responsive to the detection at the user device or to instructions generated at virtual consultation panel 130 responsive to the detection, user device 110 displays a reminder for the upcoming appointment. An upcoming appointment may be an appointment scheduled for one day, two days, several days, one week, or several weeks prior to the scheduled appointment. For example, a displayed reminder may include doctor information (e.g., “Doctor Y”) identifying the consulting surgeon, date information (e.g., “Monday, March 1”), and time information (e.g., “3:30 PM”) for the upcoming appointment, in addition to a request for confirmation of the appointment (e.g., “Will you be available at this day/time?”).

One or more selectable options can also be provided with the reminder, to confirm, decline, or reschedule the appointment. For example, a selectable confirm option (e.g., a virtual “Yes” button) and a selectable decline option (e.g., a virtual “No” button) may be displayed on a display panel of the user device. When the patient selects the “Yes” button, user device 110 sends a confirmation to virtual consultation panel 130.

Virtual consultation panel 130 and/or the user device 110 may also schedule a reminder for an imminent appointment. The reminder for the imminent appointment may be set responsive to the selection of “Yes” button at user device 110. When the schedule appointment is imminent (e.g., within five minutes, ten minutes, fifteen minutes, thirty minutes, or one hour of the scheduled appointment time), the imminent appointment may be detected by the virtual consultation application running on user device 110 and/or by virtual consultation panel 130. Responsive to the detection of the imminent appointment at the user device, or to instructions generated at virtual consultation panel 130 responsive to the detection, user device 110 displays another reminder for the imminent appointment.

For example, the displayed reminder may include doctor information (e.g., “Dr. Y”) identifying the consulting surgeon, appointment time information (e.g., “in 15 minutes”), instructions for how the patient should prepare for the imminent appointment (e.g., “Please find a private place where you are comfortable, and arrange clothing as instructed”), in addition to instructions to request connection to a virtual consultation panel 130 (e.g., “When you are ready for your consultation, please click ‘Connect’ below”).

For example, a selectable connection request can also be provided with the reminder. When the patient selects the “Connect” button, user device 110 sends a connection request to virtual consultation panel 130. Responsively, virtual consultation panel 130 generates and displays a notice with selectable connection option 1402 of FIG. 14, which can be selected to establish the video exchange between virtual consultation panel 130 and user device 110.

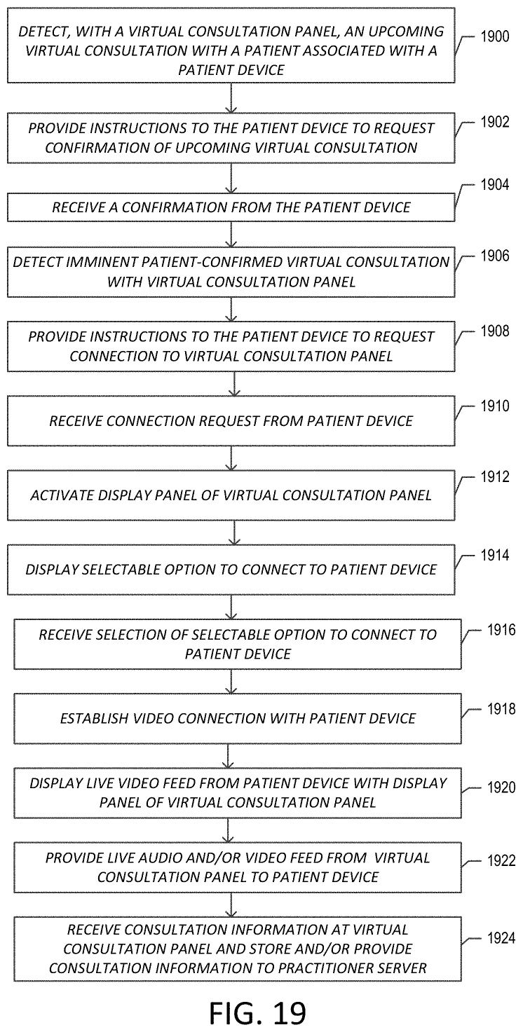

FIG. 19 illustrates a flow diagram of an example process for a virtual consultation such as a virtual surgical consultation, in accordance with one or more implementations. For explanatory purposes, the process of FIG. 19 is primarily described herein with reference to one or more devices of FIGS. 1-18 (particularly with reference to virtual consultation panel 130), which may be executed by one or more processors such as processor 436 of the virtual consultation panel 130 of FIGS. 1-18 (e.g., executing instructions stored in memory 432 such as instructions associated with virtual consultation application 440, scale processing engine 442, and video processing engine 455). However, the process of FIG. 19 is not limited to the virtual consultation panel 130, and one or more blocks (or operations) of the process may be performed by one or more other components of other suitable devices. Further for explanatory purposes, the blocks of the process of FIG. 19 are described herein as occurring in serial, or linearly. However, multiple blocks of the process of FIG. 19 may occur in parallel. In addition, the blocks of the process of FIG. 19 need not be performed in the order shown and/or one or more blocks of the process of FIG. 19 need not be performed and/or can be replaced by other operations.

In the illustrated example, at block 1900, a virtual consultation application 440 running on a virtual consultation panel such as virtual consultation panel 130 (and/or a virtual consultation application 422 running on a patient device), detects an upcoming virtual consultation with a patient associated with a patient device such as one of user devices 110. The patient is a remote patient that is located at a different location than the virtual consultation panel 130.

At block 1902, the virtual consultation panel 130 provides (e.g., via communications module 438) instructions to the patient device 110 to request confirmation of an upcoming virtual consultation. Providing the instructions to the patient device may include providing a push notification from the virtual consultation panel 130 to the user device 110, the push notification including a reminder of an upcoming appointment and a selectable confirmation option for the upcoming appointment.

At block 1904, the virtual consultation panel 130 receives a confirmation from the patient device. The confirmation may be provided by the patient device 110 responsive to selection of a confirmation option at the patient device.

At block 1906, at a time that is closer to a scheduled time of the appointment, the virtual consultation application 440 of the virtual consultation panel 130 detects an imminent patient-confirmed virtual consultation.

At block 1908, the virtual consultation panel 130 provides instructions to the patient device 110 to request connection to the virtual consultation panel. Providing the instructions to the patient device may include providing an additional push notification from the virtual consultation panel 130 to the user device 110, the additional push notification including a reminder to prepare for the upcoming appointment, and a selectable connection request to connect the user device to the virtual consultation panel.

At block 1910, the virtual consultation panel 130 (e.g., at communications module 438) receives a connection request from the patient device 110 (e.g., responsive to a selection of a connection request displayed at the user device).

At block 1912, responsive to receiving the connection request, virtual consultation panel 130 activates a display panel such as display panel 212 thereof.

At block 1914, virtual consultation panel 130 displays (e.g., in a virtual consultation application user interface) a selectable option, such as selectable option 1402 of FIG. 14, to connect to the patient device 110.

At block 1916, virtual consultation panel 130 receives a selection of the selectable option to connect to the patient device.

At block 1918, virtual consultation panel 130 (e.g., video processing engine 455) establishes a video connection with the patient device 110. Establishing the video connection may include receiving a live video feed from the patient device and/or providing a live video feed to the patient device. The live video feed may be a first live video stream including images of a patient captured by the patient device 110. Establishing the video connection may include receiving, at a virtual consultation panel, a live video stream from a remote user device, the live video stream including images of at least a portion of a body of a user of the user device.

At block 1920, virtual consultation panel 130 displays the live video feed from the patient device 110 (e.g., processed by video processing engine 455) with the display panel 212. The live video feed includes video frames, each including an image of the patient or a portion thereof, as captured by a camera associated with, and co-located with, the patient device. Displaying the live video stream with the display panel of the virtual consultation panel may include displaying the live video stream, including an actual-size representation of at least part of at least a portion of the body of the user of the user device.

The virtual consultation panel may include a mirrored outer surface, a display panel configured to project display light through the mirrored outer surface, a memory 432 configured to store instructions for a virtual consultation application 440, and one or more processors 436 configured to execute the stored instructions to cause the display panel to display the live video stream including an actual-size representation of at least part of at least a portion of the body of a user of the user device (e.g., the patient).

The virtual consultation panel 130 may also receive, from the remote user device, scale information associated with at least the portion of the user's body. The scale information may include an absolute-scale three-dimensional model of at least part of at least a portion of the body of the user, and/or may include a depth map, or other image-based scale information such as images in the video stream of a ruler or other scale indicator, and/or images of the user's hand or other reference object. In operations in which the scale information includes a three-dimensional model, the virtual consultation panel 130 may display a virtual representation of the absolute-scale three-dimensional model using scale information generated by scale processing engine 442 based on the scale information received from user device 110 and information associated with the display pixels of display panel 212. While the virtual representation of the absolute-scale three-dimensional model is displayed, the virtual consultation panel 130 may also receive an input associated with the virtual representation of the absolute-scale three-dimensional model and modify the virtual representation of the absolute-scale three-dimensional model responsive to the input. The input may include a gesture or other input for rotating or otherwise manipulating the display of the virtual representation of the absolute-scale three-dimensional model.

At block 1922, the virtual consultation panel 130 provides a live audio and/or video feed to the patient device. The live audio and/or video feed is captured by a camera such as camera 112 of the virtual consultation panel. The live audio and/or video feed may be a second live video stream including images of a consulting surgeon from the camera of the virtual consultation panel. In this way, the consulting surgeon at the location of the virtual consultation panel, and the patient at the remote location of the patient device, can interact for the virtual consultation. Displaying the actual-size representation of at least the part of at least the portion of the body of the user of the user device may include displaying the actual-size representation using the scale information received at block 1920 and/or processed by scale processing engine 442.

Providing the live video feed may include obtaining, with the virtual consultation panel, one or more images of a medical practitioner performing a surgical consultation using the live video stream, and transmitting, with the virtual consultation panel, the one or more images to the remote user device. Providing the live video feed may include receiving, with the virtual consultation panel, audio input from a practitioner performing a surgical consultation using the live video stream, transmitting, with the virtual consultation panel, the audio input to the remote user device.

At block 1924, the virtual consultation panel 130 may obtain or receive consultation information such as one or more captured still images, one or more captured three-dimensional models, one or more image annotations, one or more video notes, one or more audio notes, and/or other information generated during the consultation information by interaction with the virtual consultation panel by the consulting surgeon (e.g., by interaction with consultation tools 454 via links provided in a tools menu 1700 by virtual consultation application 440). In order to generate the consultation information using the virtual consultation panel 130, the consulting surgeon may use the virtual consultation panel 130 to provide various instructions to the patient, via the patient's user device.

For example, the virtual consultation panel 130 may provide, to the patient device 110, instructions to the patient to pinch a portion of the body of the patient in the first live video stream. The virtual consultation panel 130 may also provide, to the patient device, instructions to include scale information associated with the pinched portion of the body with the first live video stream. The scale information associated with the pinched portion of the body may be received at the scale processing engine 442 of the virtual consultation panel, and a scale indicator associated with the pinched portion of the body, such as virtual feature scale 1802 of FIG. 18, may be generated by scale processing engine 442 and displayed based on the scale information.

Still images, cropped images, cropped videos, and/or annotated images and/or videos, with and/or without the scale indicator may be generated and stored as consultation information. The received consultation information may be stored as consultation data 452 at the virtual consultation panel 130 and/or provided (e.g., via network 150) to a remote server (e.g., practitioner server 115) for storage in association with a patient file 460.

In various implementations of the virtual consultation panel 130, the virtual consultation panel 130 provides live consultation instructions to the user device 110. The live consultation instructions may include instructions spoken by the consulting surgeon and transmitted in the live practitioner video stream to the user device, and/or can include instructions generated by virtual consultation panel 130. The live consultation instructions can include instructions to the patient to move to one or more positions (e.g., front-facing, rear-facing, etc., as described herein) while in view of the camera of the user device 110, to pinch a portion of their body as described herein, and/or to provide scale information in the video stream.

For example, the virtual consultation panel 130 may provide instructions, to the user device 110, to generate instructions for the user to move into a front-facing position relative to the user device, a rear-facing position relative to the user device, a right-lateral-facing position relative to the user device, and a left-lateral-facing position relative to the user device. The instructions may include instructions to the user device 110 to display visual indicators of each of the front-facing position, the rear-facing position relative, the right-lateral-facing position, and the left-lateral-facing position. The instructions may include instructions to the user device 110 to display, using at least one depth sensor at the user device, a correctness indicator for each of the front-facing position, the rear-facing position relative, the right-lateral-facing position, and the left-lateral-facing position. The correctness indicator may be a separate visual indicator or a change in the displayed visual indicator (e.g., a change in outline thickness or color when the patient is in the correct position at the correct distance from the camera of the user device).

The live consultation instructions can include instructions to the patient to provide video of a pinch of at least a part of at least the portion of the body of the user. The virtual consultation panel 130 can also provide, to the user device, instructions for the user to provide scale information for the pinched at least part of at least the portion of the body of the user.

In various implementations, the virtual consultation panel 130 provides a request for three-dimensional (3D) information to the user device 110. The request may include a request for the user to interact with the user device to provide the 3D (e.g., scale) information, and/or may include a request by virtual consultation panel 130 for access to 3D (e.g., scale) information from one or more sensors of the user device.

Responsively the user device activates one or more 3D sensors (e.g., an infrared depth sensor or a stereoscopic imaging 3D sensor) of the user device, obtains 3D information associated with some or all of the patient that appears in the live video stream, and provides the obtained 3D information to virtual consultation panel 130. Obtaining the 3D information may include obtaining scale information associated with the live video feed/stream (e.g., using a depth sensor associated with sensor 108 of the user device, and/or using scale information captured in the live video stream). Providing the 3D information to the virtual consultation panel may include providing the scale information to the virtual consultation panel with the live video feed.

Once the virtual consultation panel 130 receives the 3D information from the user device 110, based on the received 3D information, virtual consultation panel 130 provides absolute scale information and/or other 3D information and options to the practitioner. The absolute scale information may be provided by displaying images of the patient in life size (e.g., actual size) on display panel 212, and/or may include displaying one or more rulers, scales, or calipers such as virtual feature scale 1802 of virtual patient scale 1800 of FIG. 18 on the display panel. The virtual consultation panel 130 may receive scale information including depth information from a sensor of the user device 110, and display an absolute-scale representation of the pinched at least part of at least the portion of the body of the user.

The other 3D information may include a 3D representation of the patient or a portion thereof that can be manipulated (e.g., rotated, moved, virtually pinched, etc.) by the practitioner, and/or one or more numerical features of the patient for display by the virtual consultation panel 130. The other 3D options may include options as described above in connection with, for example, FIG. 18.

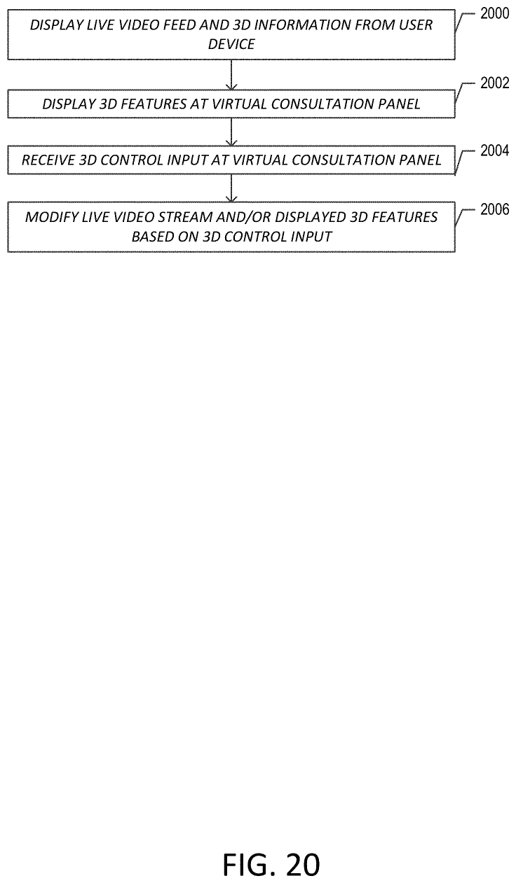

FIG. 20 is a flow chart of illustrative operations that may be performed by the virtual consultation panel using the received live video feed and the received 3D information from the user device 110.

For example, at block 2000, virtual consultation panel 130 displays the live video feed and some or all of the 3D information using display panel 212. Displaying the 3D information may include operating scale processing engine 442 and/or video processing engine 455 to overlay scale information on the displayed live video feed and/or add 3D metadata to the live video feed to facilitate 3D manipulation or visualization of the live video feed.

At block 2002, virtual consultation panel 130 displays one or more 3D features using display panel 212. The 3D features may include a virtual calipers for measuring the size of a part of the patient's body, a virtual pincher for virtually pinching a portion of the patient's body, and/or one or more additional options such as in a 3D tools menu 1700 (see, e.g., FIG. 17) and associated with virtual consultation tools 454 of FIG. 4.

At block 2004, virtual consultation panel 130 receives 3D control input associated with the live video stream. For example, the consulting surgeon may use a touchscreen feature of display panel 212, or a VR glove or other 3D controller to grab, rotate, push, pinch, or otherwise manipulate the images of the patient in the live video stream displayed in the user interface of the virtual consultation application 440, as they would manipulate a physical patient in their office for a surgical consultation (e.g., to simulate a 3D manipulation of the displayed 3D representation of the patient on the display panel).

At block 2006, virtual consultation panel 130 may modify the live video stream and/or the displayed 3D features based on the 3D control input. For example, virtual consultation panel 130 may generate an augmented reality live video stream in which the images of the patient are a 3D representation of the patient that changes as if the consulting surgeon were physically interacting with the patient's body. For example, if the surgeon pushes on a portion of the patient's abdomen, the representation of the patient's abdomen on the virtual consultation panel 130 may deform as if the surgeon were physically pushing on the patient's abdomen. The modification to the displayed representation may be generated based on physical features of the patient's body as measured using sensor 108 of the patient's own device and provided to virtual consultation panel 130 in the 3D information.

In some implementations, tactile feedback may be generated with an output device 494 (e.g., at the display panel 212 and/or by the VR controller or glove) to give the consulting surgeon the physical sensation of performing an in-office consultation.

The operations described above in connection with FIGS. 19 and 20 can be performed by the virtual consultation panel 130 in any of the configurations of FIGS. 5-10, 11, 12, 13, or other configurations of combinations of configurations described herein.

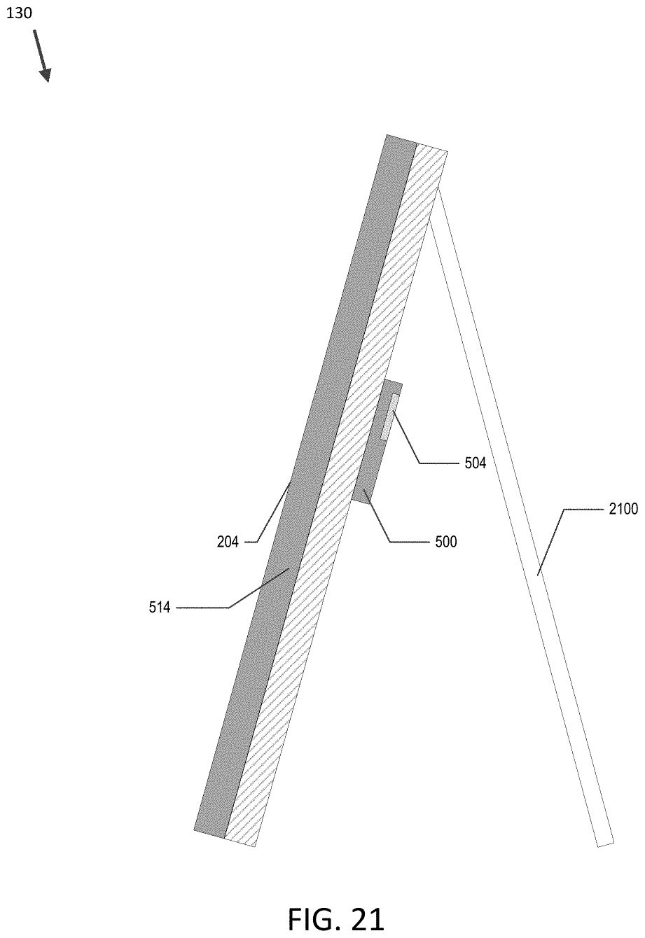

In the examples of FIGS. 5-10, virtual consultation panel 130 is provided with a mounting structure 510 for mounting the virtual consultation panel 130 to a support structure such as a wall in a doctor's office. In these implementations, virtual consultation panel 130 may be a wall-mounted or wall-embedded virtual consultation panel 130, or may be hung on another structure such as a pillar or a furniture piece. However, in other implementations, virtual consultation panel 130 may be arranged in a freestanding configuration. For example, FIG. 21 illustrates an example in which virtual consultation panel 130 (e.g., in the configuration described above in connection with FIG. 11) is provided with a structural support member implemented as a stand 2100 that allows the virtual consultation panel 130 to be freestanding within a room.

In this example, stand 2100 is an easel stand that extends at an angle relative to the rear surface of display panel 212 from a location at or near the top of the virtual consultation panel 130 to the floor, to allow the virtual consultation panel 130 to lean against stand 2100. Stand 2100 may be mounted at a permanent angle, or may be pivotable for storage of virtual consultation panel 130. Although an easel stand for a leaning virtual consultation panel 130 is illustrated in FIG. 21, it should be appreciated that other stands can be provided for virtual consultation panel 130, such as a base stand formed at the bottom of virtual consultation panel 130 that allows the virtual consultation panel 130 to stand freely and vertically in a room such as a doctor's office. The stand 2100 and/or other stands as described herein can be provided for virtual consultation panel 130 in any of the configurations of FIG. 5-10, 11, 12, or 13.

In general, the virtual consultation systems described herein allow a consulting surgeon to virtually consult with remote patients at any location at which an internet connection can be obtained. In this way, the virtual consultation systems disclosed herein utilize a novel combination and interaction of technical elements to reduce the barriers to care.

### Hardware Overview

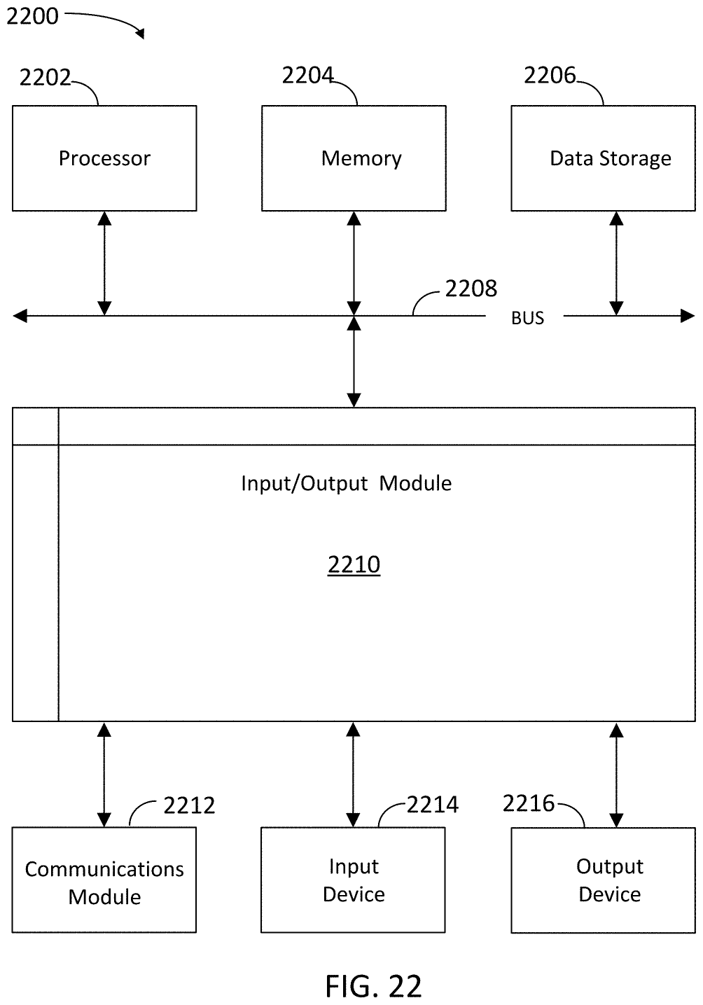

FIG. 22 is a block diagram illustrating exemplary computer system components 2200 that can be implemented in user device 110, virtual consultation panel 130, or practitioner server 115. In certain aspects, the computer system components 2200 may be implemented using hardware or a combination of software and hardware, either in a dedicated network device, or integrated into another entity, or distributed across multiple entities.

Computer system components 2200 include a bus 2208 or other communication mechanism for communicating information, and a processor 2202 (e.g., an implementation of one of processors 412, 436, or 466 of FIG. 4) coupled with bus 2208 for processing information. By way of example, the computer system components 2200 may be implemented with one or more processors 2202. Processor 2202 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system components 2200 can include, in addition to hardware, code that creates an execution environment for the computer program in question, e.g., code that constitutes processor firmware, a protocol stack, a database management system, an operating system, or a combination of one or more of them stored in an included memory 2204 (e.g., an implementation of one of memories 420, 432, or 462 of FIG. 4), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled to bus 2208 for storing information and instructions to be executed by processor 2202. The processor 2202 and the memory 2204 can be supplemented by, or incorporated in, special purpose logic circuitry.

The instructions may be stored in the memory 2204 and implemented in one or more computer program products, e.g., one or more modules of computer program instructions encoded on a computer-readable medium for execution by, or to control the operation of, the computer system components 2200, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 2204 may also be used for storing temporary variable or other intermediate information during execution of instructions to be executed by processor 2202.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 2200 further includes a data storage 2206 such as a magnetic disk or optical disk, coupled to bus 2208 for storing information and instructions. Computer system 2200 may be coupled via input/output module 2210 to various devices. Input/output module 2210 can be any input/output module. Exemplary input/output modules 2210 include data ports such as USB ports. The input/output module 2210 is configured to connect to a communications module 2212. Exemplary communications modules 2212 include networking interface cards, such as Ethernet cards and modems. In certain aspects, input/output module 2210 is configured to connect to a plurality of devices, such as an input device 2214 (e.g., a keyboard, a mouse, a touchscreen of a display panel, a microphone, a camera, a virtual-reality glove or other grasping controller, or the like) and/or an output device 2216 (e.g., a display panel such as a life-size display panel). Exemplary input devices 2214 include a keyboard and a pointing device, e.g., a mouse or a trackball, by which a user can provide input to the processor 2202. Other kinds of input devices 2214 can be used to provide for interaction with a user as well, such as a tactile input device, visual input device, audio input device, or the like. For example, feedback provided to the user with output device 2216 can be any form of sensory feedback, e.g., visual feedback, auditory feedback, or tactile feedback; and input from the user can be received in any form, including acoustic, speech, tactile, or the like. Exemplary output devices 2216 include display devices (e.g., display panel 212 and/or a display panel of a user device), such as an LCD (liquid crystal display) panel or a light-emitting diode (LED) display panel, for displaying information to the user. In some implementations, output devices 2216 include a life-sized display panel (e.g., having a height of as much as, or more than four feet or six feet, and a width of as much as, or more than, two feet or four feet) having an array of LCD or LED display elements for displaying a live video feed received from a user device. A life-sized display panel can also include a mirrored (e.g., one-way mirrored or two-way mirrored) outer surface. The display panel may include touch-sensitive components for receiving user touch input.

According to one aspect of the present disclosure, processor 2202 executes one or more sequences of one or more instructions contained in memory 2204. Such instructions may be read into memory 2204 from another machine-readable medium, such as data storage 2206. Execution of the sequences of instructions contained in main memory 2204 causes processor 2202 to perform the virtual consultation operations described herein. One or more processors in a multi-processing arrangement may also be employed to execute the sequences of instructions contained in memory 2204. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., a data network device, or that includes a middleware component, e.g., an application network device, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. The communication network (e.g., network 150) can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system components 2200 can be included in clients and network devices. A client and network device are generally remote from each other and typically interact through a communication network. The relationship of client and network device arises by virtue of computer programs running on the respective computers and having a client-network device relationship to each other. Computer system components 2200 can be, for example, and without limitation, implemented in a desktop computer, laptop computer, or tablet computer. Computer system components 2200 can also be embedded in another device, for example, and without limitation, a smart phone, a PDA, a mobile audio player, a Global Positioning System (GPS) receiver, a video game console, a server, and/or a virtual consultation panel.

The term “machine-readable storage medium” or “computer-readable medium” as used herein refers to any medium or media that participates in providing instructions to processor 2202 for execution. Such a medium may take many forms, including, but not limited to, non-volatile media, volatile media, and transmission media. Non-volatile media include, for example, optical or magnetic disks, such as data storage 2206. Volatile media include dynamic memory, such as memory 2204. Transmission media include coaxial cables, copper wire, and fiber optics, including the wires forming bus 2208. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way), all without departing from the scope of the subject technology.

It is understood that any specific order or hierarchy of blocks in the processes disclosed is an illustration of example approaches. Based upon implementation preferences, it is understood that the specific order or hierarchy of blocks in the processes may be rearranged, or that not all illustrated blocks be performed. Any of the blocks may be performed simultaneously. In one or more embodiments, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

The subject technology is illustrated, for example, according to various aspects described above. The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as configuration may refer to one or more configurations and vice versa.

In one aspect, unless otherwise stated, all measurements, values, ratings, positions, magnitudes, sizes, and other specifications that are set forth in this specification, including in the attached addendum and the claims that follow, are approximate, not exact. In one aspect, they are intended to have a reasonable range that is consistent with the functions to which they relate and with what is customary in the art to which they pertain.

It is understood that some or all steps, operations, or processes may be performed automatically, without the intervention of a user. Method claims may be provided to present elements of the various steps, operations, or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the appended claims. Moreover, nothing disclosed herein is intended to be dedicated to the public regardless of whether such disclosure is explicitly recited in the claims. No claims element is to be construed under the provisions of 35 U.S.C. § 112 (f) unless the element is expressly recited using the phrase “means for” or, in the case of a method, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Brief Description of the Drawings, and Claims of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in any claim. Rather, as the following claims reflect, inventive subject matter lies in less than all features of a single disclosed configuration or operation. The following claims are hereby incorporated into the Detailed Description, with each claims standing on its own to represent separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A virtual consultation panel, comprising:
a substrate having a first side and an opposing second side;
a display panel coupled to the first side of the substrate and comprising an array of display pixels configured to project display light through the substrate in an active mode for the display panel;
a structural support member configured to support the substrate and the display panel for viewing of the display light;
communications circuitry configured to receive a live video stream from a remote user device of a patient; and
processing circuitry configured to scale the live video stream to generate a virtual consultation view of the live video stream, for display by the display panel in the active mode,
wherein the opposing second side of the substrate comprises a mirrored outer surface that obscures viewing of the display panel, in an inactive mode for the display panel.

2. The virtual consultation panel of claim 1, further comprising a camera coupled to the opposing second side of the substrate and configured to capture an additional live video stream, using light that passes through the substrate to the camera, of a practitioner performing a surgical consultation using the scaled live video stream.

3. The virtual consultation panel of claim 1, further comprising at least one mounting bracket that attaches the display panel to the opposing second side of the substrate.

4. The virtual consultation panel of claim 3, wherein the processing circuitry and the communications circuitry are housed in a computing hub mounted to the opposing second side of the substrate.

5. The virtual consultation panel of claim 4, wherein the structural support member comprises a mounting bracket attached to the opposing second side of the substrate, and configured for mounting the substrate to another structure.

6. The virtual consultation panel of claim 4, wherein the structural support member comprises a stand configured to support the substrate and the display panel in a freestanding configuration.

7. The virtual consultation panel of claim 1, wherein the array of display pixels is formed directly on the opposing second side of the substrate.

8. The virtual consultation panel of claim 7, wherein the processing circuitry and the communications circuitry are housed in a computing hub that is mounted to a rear surface of the display panel.

9. The virtual consultation panel of claim 7, wherein the processing circuitry and the communications circuitry are disposed within a housing of the display panel.

10. The virtual consultation panel of claim 7, wherein the processing circuitry and the communications circuitry are formed on the opposing second side of the substrate.

11. The virtual consultation panel of claim 1, wherein the processing circuitry is configured to scale the live video stream to generate the virtual consultation view of the live video stream based on scale information received from the remote user device.

12. The virtual consultation panel of claim 11, wherein the processing circuitry is configured to scale the live video stream to generate the virtual consultation view of the live video stream by scaling the live video stream to an actual size of the patient based on the scale information received from the remote user device and a pixel size of the display pixels.

13. The virtual consultation panel of claim 11, wherein the processing circuitry is configured to scale the live video stream to generate the virtual consultation view of the live video stream by generating a scale indicator for display by the display panel, and scaling the live video stream to the scale indicator, based on the scale information received from the remote user device and a pixel size of the display pixels.

14. A virtual consultation panel, comprising:
communications circuitry configured to receive a live video stream from a remote user device of a user, the live video stream including images of at least a portion of a body of the user;
a display panel configured to display the live video stream, the display panel having a size that is sufficiently large to display a life-size representation of at least a part of at least the portion of the body; and
processing circuitry configured to generate the life-size representation of at least the part of at least the portion of the body based on scale information received from the remote user device.

15. The virtual consultation panel of claim 14, further comprising a camera configured to capture an additional live video stream of a practitioner performing a surgical consultation using the displayed live video stream.

16. The virtual consultation panel of claim 14, wherein the display panel comprises:
a mirrored outer surface; and
an array of display pixels that emit display light through the mirrored outer surface for displaying the live video stream in an active display mode.

17. The virtual consultation panel of claim 14, wherein the display panel comprises a touch-sensitive surface configured to receive input.

18. The virtual consultation panel of claim 17, wherein the processing circuitry is further configured to generate, for display by the display panel, an indicator of an actual size of the at least the part of the portion of the body.

19. The virtual consultation panel of claim 18, further comprising a scale processing engine configured to receive the scale information from the remote user device and generate the indicator based on the scale information.

20. The virtual consultation panel of claim 19, wherein the indicator comprises a virtual calipers that is manipulable via input to the display panel to measure one or more features of the at least the part of the portion of the body.

21. The virtual consultation panel of claim 14, wherein the processing circuitry is further configured to generate a tools menu for display by the display panel, the tools menu comprising a plurality of selectable tools for a virtual surgical consultation using the live video stream.

22. A virtual consultation panel, comprising:
a display panel;
processing circuitry configured to:
receive, from a remote user device, a live video stream including images of a patient;
receive, from the remote user device, three-dimensional information associated with the images of the patient; and
generate, for display by the display panel, a three-dimensional representation of at least a portion of the patient based on the images and the three-dimensional information; and
an input component configured to receive an input that simulates a three-dimensional manipulation of the three-dimensional representation,
wherein the processing circuitry is further configured to modify the three-dimensional representation of at least a portion of the patient based on the images, the three-dimensional information, and the input.
